(19) United States
(12) Patent Application Publication
(10) Pub. No.: US 2020/0109428 A1
(43) Pub. Date: Apr. 9, 2020

(54) PROCESS FOR PRODUCING HIGH PROTEIN BIOMASS FROM STARCH-CONTAINING CEREAL MATERIALS BY YEAST STRAINS

(71) Applicant: Le Quang Thanh, Ha Noi City (VN)

(72) Inventor: Le Quang Thanh, Ha Noi City (VN)

(21) Appl. No.: 16/593,544

(22) Filed: Oct. 4, 2019

(30) Foreign Application Priority Data
Oct. 5, 2018 (VN) 1-2018-04391

(51) Int. Cl. C12P 21/00; C12R 1/645; C12N 1/16; A23K 10/12

(52) U.S. Cl. CPC C12P 21/00; A23K 10/12; C12N 1/16; C12R 1/645

(57) ABSTRACT

A process of fermentation of starch-containing cereal materials, such as a process of producing high protein biomass from starch-containing cereal materials by yeast strains, includes following steps: producing glucose for use as a substrate from starch-containing cereal materials by liquefaction and saccharification; selecting yeast strains which produce high protein biomass and storing the selected yeast strains; and producing high protein biomass from yeast strains by using the glucose produced, optionally in combination with molasses. The produced high protein biomass is useful in animal husbandry, especially for pig, aquatic, poultry, and large-scale animal husbandry on an industrial scale. The produced high protein biomass can replace antibiotics in the livestock industry, thereby reducing imports of products from abroad.

### CROSS-REFERENCE TO RELATED APPLICATION

This application claims priority to Vietnam Application No. VN 1-2018-04391, filed on Oct. 5, 2018, which is incorporated by reference in its entirety.

### FIELD

The present disclosure relates to technology of fermentation of starch-containing cereal materials, more specifically, the present disclosure provides a process for producing high protein biomass from starch-containing cereal materials by yeast strains. The yeast-based protein product of the present invention is useful as animal feed with high economic benefits in the animal husbandry industry, such as raising pigs, aquatic products, poultry and cattle on an industrial scale, and simultaneously contributing to reducing environmental pollution due to starch processing waste.

### BACKGROUND

Demand for Food in the World and Vietnam

With a world population of 9 billion by 2050, there is strong evidence that agriculture will not be able to meet human needs and be at risk of serious food shortages (see, for example, Table 1). The production of yeast biomass is the right way to solve the difficult and catastrophic problems for mankind in the coming years. (Mekonne et. al. 2014).

TABLE 1
CONSUMING MEAT AND MILK
OF COUNTRIES IN THE WORLD

| | 1997/99 | | | 2030 | | |
|---|---|---|---|---|---|---|
| | Meat | Milk | Eggs | Meat | Milk | Eggs |
| Total | 36.0 | 78.0 | 8.7 | 45.0 | 90.0 | 10.8 |
| Developing countries | 2.5 | 45.0 | 6.5 | 37.0 | 66.0 | 8.9 |
| Developed countries | 88.0 | 212.0 | 13.5 | 100.0 | 221.0 | 13.8 |

In addition, with the recently positive development of agriculture and the prospect of future intensive farming, there will be major adverse impacts on the world's terrestrial and hydro-agricultural ecosystems. The doubling of agricultural food production in the past 35 years has been accompanied by an increase in nitrogen consumption to 6.87 times, phosphorus consumption to 3.48 times, increased irrigation water to 1.68 times, and the cultivated land area is 1.1 times. Based on the simple linear expansion of old trends, scientists predict that if global food production doubles, the demand for nitrogen and phosphorus will increase by about 3 times, the land area needs doubled irrigation, and the area of arable land increased by 18%. This will have a major impact on the diversity, composition and function of the rest of the world's natural ecosystems. The greatest impacts on scarcity of fresh water and the impact on marine ecosystems will lead to many human disasters in the future (Tilman, David 1999 May 25).

Agricultural production systems that provide food to humans contribute 19%-29% of global greenhouse gas (GHG) emissions, releasing 9,800-16,900 million tons of carbon dioxide (MtCO₂eq) in 2008 (Vermeulen et. al. 2012).

Agriculture provides food for people, but consumes a large amount of fresh water, uses a lot of land, destroys biodiversity, degrades the environment and contributes to climate change due to emissions (one third of all greenhouse gases). The process of producing single-cell protein (SCP) does not cause difficulties or disaster for mankind. Today, SCP is mostly grown with cheap crops and agricultural waste.

History of Protein Biomass Production from Single-cell Proteins (SCP)

Single-cell protein (SCP) comes from unicellular microorganisms such as unicellular algae, yeast (Candida, Saccharomyces, etc.), mycelium (Trichoderma, Fusarium, Rhizopus, etc.), and bacteria (Cellulomonas, Alcaligenes, etc.), which are used to ferment certain substrates to create functional foods and valuable biological products (Huang and Kinsella, 1986). The product is rich in protein, vitamins, essential amino acids and lipids with a high content. This protein source is able to replace the traditional protein that is slowly absorbed, the content of amino acids is unbalanced and the content of nucleic acids is high (http://scialert.net/).

According to Srivastava et al. (2008); White et al. (1954); Chen et al. (1985); Gellissen et al. (2000); Trehan et al. (1990), since 1781, people have focused on biomass fermentation. Studies of biomass fermentation technology date back to the last century, when Max Delbrück and his colleagues discovered the high value of brewer's yeast as a feed supplement for livestock. During World War II, yeast-SCP biomass was used on a large scale in Germany to combat food shortages during the war. The invention to produce SCP is often considered an important event for biotechnology in general and microbiology specifically. For example, in 1919, Sak in Denmark and Hayduck in Germany invented a method called, “Zulaufverfahren”, fermented nutritional supplements (fed-batch), by adding sugar solution continuously in during fermentation, to resist inhibiting agents, instead of mixing sugar and yeast in a batch (batch fermentation).

During the Vietnam War, the United Nations Food and Agriculture Organization (FAO) emphasized world hunger and malnutrition in 1960 and introduced the concept of a protein gap in nutrition. According to this concept, 25% of the world's population has a deficiency of the amount of protein in their diet. They worry that agricultural production will not meet the increasing demand for food, especially the protein needs of mankind. In the mid-1960s, nearly 250,000 tons of food yeast were produced in different parts of the world, and the Soviet Union alone produced about 900,000 tons of human food and animal feed in 1970.

In the 1960s, researchers at British Petroleum developed a product called “protein-from-oil” —a technology to produce yeast-protozoan protein fed with n-paraffin, a product side of the refinery. The initial research was carried out by Alfred Champagnat at Lavera refinery in France. The pilot-type industrial plant started operations in March 1963, with the construction of a similar industrial plant at Grangemouth oil refinery in England.

Problems of SCP production were posed in 1966 by Carroll L. Wilson of MIT. The idea of “food-from-oil” became quite popular in the 1970s and was awarded the “UNESCO Science Prize” in 1976. Since then, a number of yeast-producing facilities with paraffin-fed supplementation method were formulated in several countries. Product SCP was created for poultry use.

The Soviet Union particularly responded to the expansion of production of “BVK” (belkovo-vitaminny kontsentrat, namely “concentrated protein-vitamin”), with a factory next to its refinery in Kstovo (1973) and in Kirishi (1974). The Soviet Industrial Microbiology had eight factories of this type in 1989. However, due to concerns about alkalinity toxicity in SCP and pressure from environmental protection movements, the government decided to shut them down, or convert them to some other microbiological process.

The yeast product, Torprina, is produced from C. lipolytica and C. tropicalis that have been commercialized to replace fishmeal and skimmed protein in protein-rich foods. The substrate for yeast protein production of Toprina is C₁₂-C₂₀ alkane from the petroleum industry. In the late 1950s, British Petroleum built a 16,000 tons/year industrial plant in France and a 4,000 tons/year industrial plant in the UK. In 1977, SCP production was halted due to the rise in oil prices and low soybean prices, making the product difficult to compete.

Mushroom products are manufactured by Amoco Company in the U.S. from Torula yeast strains (today called Candida utilis). However, the enzyme substrate, so-called Torutein, used for fermentation to produce Torula biomass, is ethanol which is very expensive. The government has certified Torutein products with a protein content of 52% and a low methion content. Torutein was commercialized, allowing replacement of meat, milk, and eggs, but failed because the price of soy in the U.S. was very low.

Types of microorganisms which ferment biomass and substrate sources are described in Richard I. Matelles and Steven, 1978.

Types of microorganisms:

Yeast: S. cerevisiae, Pichia pastoris, C. utilis, Torulopsis, Geotrichum candidum, Kluyveromyces marxianus, Endomyces vernalis, Hansenula anomala, Hansenula suaveolens, C. arbores, C. tropicalis, Mycotorula lipolytica, Mycotorula japonica, Torulopis utilis, Torulopis utilis var major, Torulopsis utilis var thermophilis, Monilia candia, and Oidium lactic;

Mycelium: Aspergillus oryzae, Fusarium venenatum, Sclerotium rolfsii, Polyporus, Trichoderma, and Scytalidium acidophilum;

Bacteria: Rhodobacter capsulatus; and

Algae: Chlorella and Spirulina.

For producing protein from yeast-like hydrocarbon sources, the following criteria must be met:

able to assimilate many different sources of carbon, especially pentose (xylose, arabinose) and organic acids;

can develop well on high concentrations of reducing agent (sugar concentration);

able to grow fast, have high resistance to CO₂ concentration;

have high yields of high biomass containing valuable nutrients (high protein content, high amounts of essential amino acids, vitamins, etc.);

have relatively large cell size for easily separating by centrifugation; and

withstand relatively high temperatures, and low changes in environmental pH.

In the production of protein biomass, strains of four genera: Saccharmyces, Candida, Kluyveromyces and Torulopsis are commonly used. The transformation ability of these four varieties is very high and diverse, and the technology is relatively simple. Many strains of SCP can grow on different substrate sources.

Strains of S. cerevisae, Candida utilis, and Kluyveromyces marxianus have different origins from France, Canada etc., and are currently being marketed as supplements to feed as supplementary protein or probiotic. Strains of S. cerevisae can be combined with Lactobacillus (L. delbruckii and L. coryneformis) to ferment cereals, and the protein content can be increased by 8.2% (G., 2006).

Protozoan proteins from yeast, algae, and micro-fungi are also processed to produce flavor enhancers and food odorants (www.bpfoods.com). The Quorn™ product, containing myco-protein (hwww.quorn.com), first produced in 1964, makes human food from F. venenatum A3/5 on a starch basis and agricultural by-products. Currently, the continuous fermentation technology used for production of this unicellular protein, with controlled cell density to produce a meat-like flavored product, has been commercialized. In addition to its beneficial effects on digestion and immunity, the microbial strains added to feed are also a source of highly quality protein.

Candida Utilis or C. Utilis

Candida utilis is an important industrial microorganism. It is used to produce a number of useful biological materials, such as glutathione, amino acids and enzymes. It is also a promising source of unicellular protein through large-scale production from sugars, such as molasses and sulfite alcohols.

The dilution of substrates medium, such as sugar, is an important parameter affecting the production of Candida utilis biomass. Temperature, oxygen transmission rate (OTR), pH and ethanol concentration of about 50 g/L during fermentation were monitored. The produced ethanol affects the cell growth rate, and the conversion of acetate into H₂O and CO₂, resulting in insufficient energy for biomass biosynthesis. Ethanol levels begin to inhibit cell growth related to OTR dissolved oxygen levels (Straskrabová, V et al. 1980). Various ways to deliver pure oxygen have been tested for ethanol-containing media.

The results obtained from continuous culture media show a steady state, showing that fed ethanol and oxygen provide significant, apparent effects on cell growth rate, ethanol catabolism rate, acetate production and biomass productivity and productivity (Paca, J. 1982; www.efsa.europa.eu). The growth and development of C. utilis yeast occurs at a wide range of temperature from 18 to 37° C., in a glucose-rich media. Under conditions of saturated oxygen, the ability to increase biomass doubles after 60-80 minutes of culturing. Under poor oxygen conditions, the fermentation will produce ethanol. C. utilis has many useful biological properties (www.efsa.europa.eu) and has been used very effectively in the production of protein for animal feed.

Kluyveromyces Marxianus

K. marxianus is also known as Saccharomyces vanudenii or Kluyveromyces vanudenii. This species was first described in the Saccharomyces genus as S. marxianus, discovered by Danish scientist, Emil Christian Hansen, from beer. He named the species after biologist, Louis Marx in Marseille, who first isolated it from grapes. This species was transformed into the Kluyveromyces genus by van der Walt in 1956. Since then, 45 species have been recorded in this genus. The closest relation of Kluyveromyces marxianus is the Kluyveromyces lactis yeast, commonly used in the dairy industry. Both Kluyveromyces and Saccharomyces are considered part of the “Sacchromyces complex”, a sub-branch of Saccharomycetes.

Using the 18S rRNA gene sequence suggests that K. marxianus, K. aestuarii, K. dobzhanskii, K. lactic, K. wickerhamii, K. blattae, K. thermotolerans, and K. waltii form a separate branch of separate ancestors from the central branch in the Kluyveromyces genus. In this complex, two types are identified based on the presence in certain taxa of an entire genome-duplication event: two branches are called pre-whole genome duplication (WGD) and after WGD. The Kluyveromyces species were associated with the first of this species while the Saccharomyces species belonged to this species later. In order to distinguish these branches, it must be based on the WGD event to explain why, although the two species are closely related, the basic difference exists between them.

K. marxianus colonies are cream to brown in color, and yeast cells are spherical, elliptical or cylindrical with a size of 2-6×3-11 μm. K. marxianus is heat-resistant, and has a high ratio of growth at 40° C. (104° F.). Kluyveromyces marxianus is an aerobic enzyme that is able to metabolize respiratory fermentation, generating energy simultaneously from both processes that are aerobic through the TCA cycle and the ethanol fermentation. The species also ferments on media substrates of inulin, glucose, raffinose, sucrose and lactose into ethanol. No assimilation of nitrate; no use of ethylamine, cadaverine, or lysine; and the use tryptophan as a nitrogen source occur. Growth requires vitamins such as biotin and niacin, with variable growth on high glucose, with 10% NaCl.

Cells are reproduced by sprouting, and the spores are smooth spores ranging from pea-shaped spheroidal or crescent-shaped spheres. Adult spores often contain lipid droplets. Conjugation immediately occurs upon spore formation.

Gametes have a passive motile form (phytoplankton), with 1-4 spores per ascus. Growth in a liquid medium occurs with cells that settle down, round in shape, and thin films can be formed.

The number of chromosomes is between 4-18. K. marxianus is used commercially to produce lactase enzymes and as a binder for animal feed and pet food, and as a source of ribonucleic acid in pharmaceuticals.

K. marxianus is found in grape juice but mostly in milk, cheese and other dairy products. It is able to withstand high temperatures, growing on NaC—YM medium. K. marxianus is able to ferment sugars in high-temperature environments (up to 45° C.) including grape juice, in some commercially complex preparations for flavor. It is sensitive to a number of factors such as SO₂, sorbate, DMDC, pH, and acid. However, it is insensitive to ethanol and temperature (Davis U C. 2002).

The new fermentation properties of the heat resistance of K. marxianus var marxianus have been identified to assess its usability in ethanol production. Stable growth is not observed under anaerobic conditions, even in the presence of unsaturated fatty acids and sterols. Maximum ethanol concentration of 40 g/L occurs at 45° C., with an initial specific ethanol production rate of 1.7 g/hour. This is observed at ethanol concentrations below 8 g/L and under limited oxygen conditions. This strain is not suitable for ethanol production due to low ethanol resistance at low concentrations and low growth under oxygen-limited conditions. This strain has many advantages in industrial biomass production (Hack C J et al. 1998) with the ability to generate rapid cell biomass (Bajpai P et al. 1986).

K. marxianus grows rapidly, and produces large biomass used as a biocatalyst to reduce the impact of nitrogen-containing waste. Ammonium sulfate is used as a source of nitrogen (this salt causes acidification of the environment with sulfate). Using (NH₄)₂CO₃ instead of (NH₄)₂SO₄ is not useful because increasing (NH₄)₂CO₃ raises the pH to inhibitory levels. K. marxianus DSM 5422 assimilates urea as an alternative nitrogen source. The use of urea will reduce salt load, less inhibition of yeast cell growth, and will reduce environmental impact (Christian L et al. 2015).

Ability to Produce Biomass of K. Marxianus Yeast

The growth ability of K. marxianus is very high and can grow 100 times in 16 hours, from 0.7 g/L to 70 g/L (Érika Durão Vieira et al. 2012). Similarly, Christian Loser et. al. (2015) also studied the possibility of increasing the cell biomass of K. marxianus strain on the whey media discharged from a milk processing plant, using Kluyveromyces marxianus DSM 5422 to ferment, along with the addition of an inorganic nitrogen source (urea, ammonium sulfate), which resulted in a biomass of 82 g/L. Similarly, Sharbadeb Kundul et al. (2012) also used whey from a milk processing plant to ferment. Their research is optimizing the factors (whey concentration, temperature, pH, etc.) to obtain the best yeast biomass in the laboratory by using of K. marxianus MTCC 4059 strains. Results show that production is the highest of 8.38 g/L at 3% lactose content; pH=7 (if pH=6, the biomass of yeast is 1.45 g/L; if pH=8, the biomass of yeast is 1.1 g/L), the temperature is 30 degrees C., and the time is 29 hours.

Gustavo Graciano Fonseca (May 2007) conducted the research project “Physiology of the yeast Kluyveromyces marxianus during batch and chemostat cultures with glucose as the sole carbon source”, using the yeast Kluyveromyces marxianus ATCC 26548 in a glucose medium, with pH=5, and the results also show that the biomass yield on glucose is 0.51 g. This means that for every 100 g of glucose, the process will produce 51 g of biomass, the protein content of the dry matter of the biomass is 54.6%, and the ability to digest oxygen is 11.1 mmol/g.

As mentioned above, strains of K. marxianus and Candida utilis have many biochemical characteristics, which are fast growth and rapid biomass production, and high efficiency, due to the ability to produce ethanol lower than other yeast strains. Simultaneously, these strains can use a lot of carbon sources, inorganic nitrogen sources (excess waste in agriculture and industry), contributing to reducing environmental pollution, increasing collection for producers and recycling waste materials. It has been widely used in the world for its safety, biomass production efficiency and nutritional quality in food and in food processing.

Saccharomyces Cerevisiae or S. Cerevisiae

S. cerevisiae was described by Meyen (1938): ovoid, oval, size of 3-6 μm, proliferation, white colonies, round edge, convex, glossy surface, diameter of 1-2 mm. S. cerevisiae can use sugar, malt, high sugar, etc., cannot directly use starch, cellulose, hemicellulose, etc., optimal developing at 28-30° C., poor reproducing above 43° C. and below 28° C., pH about 4.5-5.6. Yeast cells are very nutritious including nucleonic acids, proteins (50-75%), carbohydrates (4-13%), without lipids, vitamins, etc.

Growth and development of S. cerevisiae yeasts occur at a wide temperature range from 18-37° C., with glucose-rich media. Under conditions of saturated oxygen, the ability to increase biomass doubles after 60-80 minutes of culturing. In an oxygen-poor environment, the fermentation produces products such as ethanol (used in the manufacture of beverages). Glucose-containing media supplement nitrogen sources to produce high protein biomass (Vu et al. 2009a; Vu et al. 2013).

Strains of S. cerevisia are used in food and animal nutrition very effectively (www.efsa.europa.eu). Strains of S. cerevisia are heat-resistant, and yeast cells live well and are stable in small intestine and rumen. S. cerevisiae is used as a probiotic to stabilize intestinal microflora, enhance digestibility of food, and has been used for 12 different species of animals with good effects. S. cerevisiae increases milk yield by 3 L/day, culminates in early and higher lactation, maintains long-term milk intake, increases protein content by 4.3%, and increases fat by 9.8%. For pigs, the ability to increase meat, milk production and quality is improved. S. cerevisiae plays a role in improving the immune system, reducing nail inflammation, sore feet, improving health, and improving food absorption.

S. cerevisiae is responsible for regulating the redox in the rumen and stomach, stabilizing pH, increasing fiber digestion, reducing problems in young animals, and increasing milk production and quality. The β-glucan in yeast is high in content, thereby boosting immunity, enhancing the activity of macrophages and stimulating the secretion of more cytokines. The β-glucan helps reduce the food conversion ratio, stimulates digestion, prevents intestinal diseases, bacterial and viral infections, gives appetite (Reed and Nagoda, 1991; Temelli and Burkus, 2000), and reduces antibiotic use. The yeast cell wall has a strong ability to adsorb mycotoxins (Uscanga et al., 2003; Uscan B A, 2003) (FIG. 1a-b).

Advantages and role of single-cell protein (SCP) products include:

labor cost is much less than agricultural production;

can be produced in any location, not affected by weather and climate, industrial processes, and has easy mechanization and automation;

produces microbial biomass which requires less fresh water than cultivation, to create a protein biomass together, and it is possible to use this method for dry areas that cannot be cultivated or nutrient-poor soils;

has high productivity: microorganisms have strong reproduction speed, fast growth ability: (algae: 2-6 hours, yeast: 1-3 hours, bacteria: 0.5-2 hours), so in a short time, a huge amount of biomass can be collected, and this time is indicated in a time in hours, and in animals and plants, in months or decades, which also allows the selection of high-yielding strains and good nutrition quickly and easily compared to breeding;

uses cheap and high-efficient raw materials, which are usually waste products, by-products of other industries such as molasses, suites, alkanes, petroleum paraffin, methanol, methane, ethanol and sugars, etc., even the effluent from a certain production processes, with high conversion efficiency: carbohydrates are metabolized up to 50%, and carbon hydride up to 100% into the dry matter of the cells;

has very high protein content in cells: in bacteria is 60-70%, in yeast is 40-60% dry matter, etc., and this content depends on the species and is influenced by culture conditions, with the protein content here containing only protein, not including the non-protein nitrogen component when determined by the total nitrogen method of Kjeldal, such as nucleic acids, or the peptides of the cell component;

has high quality protein: many amino acids in microorganisms with high content, such as in the products of meat, milk and protein compared to plants, and microbiological protein is particularly rich in lysine, which is a great advantage when supplementing food and livestock, because food often lacks this amino acid, while in contrast, the content of sulfur-containing amino acids is low;

has some microorganisms that can synthesize vitamins and nutrients that cannot be produced or not produced in large quantities by the industrial plant, including vitamin B12, and this yeast also contains β-glucan which enhances body immunity;

has this yeast which produces lactase enzyme, in which some people and animals lack this intestinal lactase and this yeast in the intestinal tract, and develops and produces the mucosal lactase enzyme that is responsible for hydrolyzing lactose disaccharide in milk; and

has yeast biomass which is easier to separate than bacteria, while in addition, microorganisms capable of growing at high densities will produce high yields, growing well at high temperatures (with thermophilic and heat-resistant properties), which will reduce cooling costs in production, less susceptible to contamination etc., with strains of microorganisms that use inexpensive, metabolized carbon sources as much as possible will be used in production, and therefore, yeast is mainly used in unicellular protein production.

Disadvantages of SCP:

has rapidly growing microorganisms such as bacteria and yeast which have a high concentration of nucleic acids, especially RNA, and the level must be limited in diets below 50 g per day, or overdose will result in increased uric acid concentration in plasma, which can cause gout and kidney stones, with uric acid which can be converted to allantoin, which is excreted in the urine, while the elimination of nucleic acids is not only necessary for animal feed but also is essential in human food, and keeping the temperature at 64° C. will inactivate the protease and allow RNase to remain active, such that RNase acts by hydrolyzing RNA to release nucleotides in a microbial culture medium; and

acts like plant cells, since cells of some microorganisms such as algae and yeast contain undigested components, such as cellulose, and thus, the cells of some types of SCPs need to be broken down to release the cytoplasm inside the cells so that humans and animals can digest easily.

Some types of SCP also have an unpleasant taste.

Depending on the type of SCP and the culture medium, care must be taken to avoid contamination by other microorganisms that cause the production of certain toxins such as mycotoxins or cyanotoxins. An interesting approach to solving this problem has been proposed with the Scytalidium acidophilum fungi that grow at low pH levels. This allows the hydrolysis of paper waste into sugar to be used as a low-cost yeast culture medium.

Some yeasts and filamentous mushrooms used to produce proteins contain low levels of methionine.

Products and Chemical Composition of Yeast

Yeast is a single-cell protein source that has many advantages compared to traditional protein sources (soybeans, meat and fish etc.) because of the short growth and development time, high protein biomass, continuous production, regardless of weather conditions, and developing on cheap available materials (by-products of the sugarcane industry, the paper industry, starch processing and fruit processing, etc.). Yeast fermentation also produces vitamins such as riboflavin, thiamin, and noxins and essential amino acids like alanine, glutamic acid, histidine, lysine, isoleucine, valine, tryptophan, phenylalanine. Therefore, the livestock industry needs to strongly exploit the source of microbial fermentation protein, contributing to alleviating the difficulties of the current shortage of protein materials.

Chemical Composition of Yeast Protein

Yeast is rich in protein and vitamins, especially type-B vitamins.

Slurry biomass contains 20-25% dry matter while carbon is 40-50%, nitrogen is 7-10% (about 40-60% protein), hydrogen is 5-7%, O is 25-30%, inorganic substances is 5-10% (of which phosphorus and potassium are 95-97% of the total ash, and the other elements are Ca, Mg, Al, S, Cl, Fe, a trace amount of elements including Mn, Zn, Mo, Bo, Co etc.). Yeast cells also contain most essential substances for life such as carbohydrates, lipids, enzymes, nucleic acids, etc.

In terms of properties, the yeast protein is almost identical to the animal protein. Yeast protein contains about 20 non-replaceable amino acids. The amino acid composition of yeast is more balanced than that of wheat and other cereal grains, slightly less than milk, fish meal, meat meal and general animal products (Tables 2 and 4). No plant or animal product contains in its content the same amount of specific effects as yeast.

TABLE 2
CHEMICAL COMPOSITION OF SOME
UNICELLULAR MICROORGANISMS

| Composition | Mycelium | Algae | Yeast | Bacteria |
|---|---|---|---|---|
| Protein | 30-45 | 40-60 | 45-55 | 50-65 |
| Lipid | 2-8 | 7-20 | 2-6 | 1-3 |
| Ash | 9-14 | 8-10 | 5-10 | 3-7 |
| Nucleic acids | 7-10 | 3-8 | 6-12 | 8-12 |

### (Miller and Litsky (1976))

TABLE 3
ESSENTIAL AMINO ACIDS IN SOME IMPORTANT
MATERIALS

| | | Egg- | S. | C. | P. |
|---|---|---|---|---|---|
| Amino acids | Barley | white | cerevisiae | lipolytica | notatum |
| Lysine | 2 | 6.5 | 7 | 7.8 | 3.9 |
| Threonine | 2.9 | 5.1 | 4.8 | 5.4 | — |
| Methionine | 1.5 | 3.2 | 1.7 | 1.6 | 1.0 |
| Cystine | 2.5 | 2.4 | — | 0.9 | — |
| Tryptophan | 1.1 | 1.0 | 1.0 | 1.3 | 1.25 |
| Isoleucine | 3.3 | 6.7 | 4.6 | 5.3 | 3.2 |
| Leucine | 6.7 | 8.9 | 7.0 | 7.8 | 5.5 |
| Valine | 4.4 | 7.3 | 5.3 | 5.8 | 3.9 |
| Phenylalanine | 4.5 | 5.8 | 4.1 | 4.8 | 2.8 |

Some types of unicellular protein for feed can be fed directly without treatment, while others need to go through some basic processing steps to increase the ability to absorb, and to create appropriate flavors of the product. For single-cell protein products from yeast, the most common use of the mechanism of self-cell breakdown by endogenous enzymes with heating steps is to ferment the yeast fermentation up to 45-50° C. for 24 hours at pH 6.5. Under these conditions, intracellular enzymes hydrolyze partially the cell wall and proteins into a simple peptide that is more easily absorbed. This method should be strictly controlled, to avoid hydrolysis reactions to create peptides with unpleasant taste, affecting the ability to use the product (Castrillo, 2002). Some other treatments, such as the use of glass beads for grinding, ultrasonic or cold grinding, are only suitable for small-scale manufacturing processes.

Intracellular components (nucleic acid): The nucleic acid content in yeast cells is 5 times higher in humans and animals. When single-cell protein is used as animal feed, elimination of nucleic acid is not necessary. In humans, if using below 100 g/person/day, the nucleic acid content does not need to be treated; if using above 100 g of single-cell protein/person/day, the nucleic acid content should be processed and eliminated. When nucleic acids enter the animal's digestive tract, the nucleosides are further broken down into purines and pyrimidine groups, creating uric acid. The accumulation of uric acid beyond the kidneys' ability to excrete leads to the formation of crystalline uric acid that accumulates in muscles, cartilage tissue and kidney cysts, causing gout and kidney stones. Alkaline hydrolysis helps to reduce RNA content but also reduces the nutritional value of the product. Some single-cell protein products from yeast, at 60° C. in the presence of pancreatic ribonuclease, reduce nucleic acid from 9% to 2% (Solomons, 1983). In addition to the nucleic acid composition, a number of allergenic and mutagenic indicators are also strictly tested in single-cell protein products used as food.

The single-cell protein is usually used at 8-12% in the diet of cattle and poultry and produces good results if the diet balances energy and vitamins. The unicellular protein derived from yeast has been used successfully in animal feed, poultry and aquaculture since the 1950s.

TABLE 4
AMINO ACID CONTENT AFTER HYDROLYSIS
OF YEAST PROTEIN S. CEREVISAE

| | Content (%) | Content (%) |
|---|---|---|
| | according to protein | according to |
| Amino acids | (Nx6.25) | cell biomass |
| Lysine | 7.04 | 2.97 |
| Methionine | 2.1 | 0.9 |
| Cysteine/Cystine | 1.42 | 0.6 |
| Threonine | 4.9 | 2.07 |
| Tryptophan | 1.26 | 0.53 |
| a-alanine | 8.15 | 3.43 |
| Leucine | 9.99 | 4.21 |
| Isoleucine | 5.2 | 2.45 |
| Phenylalanine | 5.1 | 2.23 |
| Valine | 6.3 | 2.66 |
| Aspartic acid | 9.68 | 4.08 |
| Glutamic acid | 13.67 | 5.76 |
| Glycine | 4.8 | 2.02 |
| Histidine | 2.55 | 107 |
| Proline | 5.44 | 2.29 |
| Serine | 4.5 | 1.91 |
| Arginine | 4.24 | 1.79 |
| Tyrosine | 4.21 | 1.78 |
| Total (%) | 100.55 | 38.67 |

Sources of Fermentation Substrates

1. High Power Source

Methane, methanol, C₁₂-C₂₀ alkanes (wax form) and oils were of interest to produce SCP biomass by bacteria (British Oil Company) and Candida lipolytica and C. tropicalis yeast (Riviere, 1977). However, because of toxicity, the production of SCP from petroleum was stopped (in 1977 in Italy), and so far no industrial plants have produced petroleum proteins.

Methylomonas methanica uses methane and nitrogen sources (nitrate salts, ammonium salts) as substrates to produce SCP, but this technology faces many difficulties. The M. methtlotrophus strain uses methanol to produce SCP more effectively in the UK. Methanol has many advantages over n-paraffin, methane, and other types of carbohydrates, causing no toxicity issue.

2. The Source of Waste

Using waste substrates (cellulose, lignin, hemicellulose, starch, etc.) to produce SCP is a new trend adopted by Japan and some Eastern countries, both having a source of protein biomass and reducing environmental pollution (Callihan and Clemmer, 1979).

Wood is treated with CaSO₃ and SO₂ to release lignosulfonate, hemicellulose, then is saccharified to make substrates for S. cerevisia and some other yeasts. The protein content produced by the fungus Paecilomyces variotii exceeds 55% (w/w) (in Finland) with a project of producing 7000 tons/year (Oura, 1983).

Enzyme mixtures (endoxenlulase, cellobiohydrolase, cellobiase) are commonly used for fiber metabolism. Chaetomium cellulolyticum fungus has the ability to generate a cellulose decomposition enzyme, and it uses cellulose to produce protein biomass. The amino acid composition of proteins from this strain is similar to that of amino acids in soybeans, and is better than the proteins from T. viride strains (Bhalla et al., 2007).

Endomycopsis fibuligira strains produce amylase and C. utilis strains for fermenting cereals (Moebus and Teuber, 1983). In 1956, Pioneered Company in France implemented a project from whey using the strain K. marxianus (formerly known as K. fragilis) to assimilate lactose (Oura, 1983; Moulin et al., 1983). Trichoderma strains ferment from waste from coffee production, Cellulomonas ferment bagasse, and Thermoactinomyces ferment animal wastes, used to produce SCP in Guatemala (Humphrey, 1975).

Molasses

Molasses has nutritional components (%): total sugar 48-56, other organic substances 9-12, proteins 2-4, potassium 1.5-5, Ca 0.4-0.8, Mg 0.06, P 0.6-2. Molasses also contains (mg/kg): biotin (vitamin H) 1-3, vitamins (thiamine, riboflavin, pyridoxine, folic acid, biotin, pantothenic acid), pantothenic acid 15-55, Inositol 2500-6000, and trace amounts of elements including Zn, Mn, Cu, B, Co and Mo. Vitamin H (biotin) stimulates growth for most yeasts (White, 1954).

Molasses is a good substrate for microbial fermentation to create valuable products. One hundred kilograms of raw cane or sugar beets create 3.5 to 4.5 kg of molasses (Oura, 1983). In Vietnam, total sugarcane area is 300,000 ha, annual sugarcane output reaches nearly 20 million tons, sugar production reaches 1 million tons and molasses reaches 700,000 to 900,000 tons.

Yeasts use molasses directly to create biomass and their metabolism, without any hydrolysis process (because yeasts can hydrolyze sucrose to make glucose and fructose). S. cerevisiae can be directly used for growth, development, and biomass production under oxygen conditions (Callihan and Clemmer, 1979).

Vu et. al. (2009a) used molasses and corn-starch byproducts for liquid fermentation with a substrate and optimized biomass creation medium (180 g biomass/L).

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

An object of the present invention is to provide a process for producing yeast biomass and probiotic protein from starch and molasses, according to modern technological processes, automating and can be applied in industrial scale.

Another object of the present invention is to provide a process for producing protein-rich products with high fermentation technology from cereals and molasses to replace soybeans and other imported protein sources in the production of animal feed.

### Solution to the Problem

To achieve the above purposes, according to one aspect, the present invention provide a process for producing high protein biomass from starch-containing cereal materials by yeast strains, with this process comprising the following steps:

i) producing glucose for use as a substrate from the starch-containing cereal materials by liquefaction and saccharification, including:

cleaning the starch-containing cereal materials and grounding;

carrying out liquefaction by raising the temperature to 85° C. and adjusting the pH to 5.5, then adding the enzyme α-amylase to carry out the liquefaction within two hours;

performing saccharification by lowering the temperature to 65° C. after two hours of liquefaction, and adjusting the pH to 4.5, then quantifying the α-glucoseamylase enzyme immediately after cooling and adjusting the pH to 4.5, before the saccharification process takes place;

conducting glucose content analysis by qualitative test using reagents such as iodine to determine the end of the saccharification process; and

separating glucose from the residue and stores the resulting sugars;

ii) selecting yeast strains which produce high protein biomass and storing the selected yeast strains, wherein:

selecting the yeast strains, which are capable of fermenting biomass, include S. cerevisiae, Pichia pastoris, C. utilis, Torulopsis, Geotrichum candidum, Kluyveromyces marxianus. Endomyces vernali, C. arbores, C. tropicalis, Torulopis utilis, Torulopis utilis var major, S. cerevisiae, Kluyveromyces marxianus, C. tropicalis; Monilia candia, Pichia pastoris, L. curvatus, L. reuteri, L. plantarum, L. parapentarum, and L. Pentosus;

culturing on a YPG agar medium including 10 g/L of yeast extract, 20 g/L of peptone and 20 g/L of glucose at 30° C., pH of 5.5, within a range of 18 to 24 hours, to refine each strain;

fermenting on the YPG agar medium including 10 g/L of yeast extract, 20 g/L of peptone and 20 g/L of glucose at 30° C., pH of 5.5, within 24 hours;

sampling for glucose analysis by High Performance Liquid Chromatography (HPLC);

selecting strains, preferably one or two, with the greatest biomass fermentation capacity when the residual glucose content is determined to be between 4-6 g/L;

genetic sequencing to identify yeast strains based on gene maps; and

storing the selected strains; and

iii) producing high protein biomass from yeast strains of step ii) by using glucose produced in step i), including:

selecting the yeast strains of step ii) which are propagated through three levels, level 1, level 2 and level 3, in the fermentation vessel in the corresponding medium, wherein:

level 1 and level 2 fermentation are carried out in the YPG medium including 10 g/L of yeast extract, 20 g/L of peptone and 20 g/L of glucose, at 30° C. and pH of 5.5;

level 3 fermentation is carried out in a medium selected from the group consisting of: glucose produced in step i) and glucose produced in step i) in combination with molasses, to sufficiently produce a variety of cells having a minimum cell density of 10⁸ cells/mL for the propagation step in the fermentation stage on the industrial scale, wherein:

glucose is maintained at 200 g/L, or glucose in combination with molasses at a concentration of 100 g/L for glucose and 180 g/L for molasses to optimize the fermentation;

fermenting on an industrial scale, in which this step also goes through five levels of fermentation: level 1, level 2, level 3, level 4 and level 5, with the fermentation substrate source selected from the group of glucose produced in step i) at a concentration of 200 g/L and glucose produced in step i) in combination with molasses at a concentration of 100 g/L for glucose and 180 g/L for molasses; and

analyzing of glucose, sucrose and ethanol content in a fermentation solution after the fermentation process to determine the end of the process in order to obtain the most effective biomass.

In the preferred embodiment of the process for producing high protein biomass according to the present invention, the starch-containing cereal materials are selected from the group of cassava, maize, wheat and rice.

In another preferred embodiment of the process for producing high protein biomass according to the present invention, the pH adjuster is selected from the group consisting of a H₂SO₄ solution and a 20% NaOH solution.

In still another preferred embodiment of the process for producing high protein biomass according to the present invention, in step i) the added α-amylase enzyme is calculated according to the starch content of 0.02% during the liquefaction.

In still another preferred embodiment of the process for producing high protein biomass according to the present invention, the end of the process of sugarification in step i), based on an analysis of the glucose content, is determined when the glucose content is asymptotic to the theoretical glucose content, preferably approximately 110% compared to starch.

In still another preferred embodiment of the process for producing high protein biomass according to the present invention, the strains with the greatest biomass fermentation capacity in step ii) are Kluyveromyces marxianus and C. utilis, with a cell density of 2.5×10⁸ cells/mL and 5.75×10⁸ cells/mL, respectively.

In still another preferred embodiment of the process for producing high protein biomass according to the present invention, the strain with the greatest biomass fermentation capacity in step ii) is Kluyveromyces marxianus.

In still another preferred embodiment of the process for producing high protein biomass according to the present invention, the strain with the greatest biomass fermentation capacity in step ii) is Kluyveromyces marxianus KJ830981.1.

In still another preferred embodiment of the process for producing high protein biomass according to the present invention, storage of the selected strain in step ii) is carried out in an environment of glycerol 30% at a temperature of approximately −70° C.

In still another preferred embodiment of the process for producing high protein biomass according to the present invention, the molasses for fermentation is treated to de-color and remove colloid.

In still another preferred embodiment of the process for producing high protein biomass according to the present invention, the colloid removal involves removing a colloidal system containing high viscosity protein and pectin.

In still another preferred embodiment of the process for producing high protein biomass according to the present invention, the colloid removal process is performed using a method selected from the group consisting of a chemical method and a mechanical method.

In still another preferred embodiment of the process for producing high protein biomass according to the present invention, the chemical method is performed by using H₂SO₄ and lime to precipitate colloid, form a binding with salt and decomposed protein.

In still another preferred embodiment of the process for producing high protein biomass according to the present invention, the mechanical method is carried out by:

i) diluting molasses with water in the ratio of molasses/water (ton/m³) of about 1.0:0.7;

ii) adding CaCl₂ with the ratio of CaCl₂/molasses (kg/ton) of about 0.9:1, stirring for 30 minutes and depositing for 30 minutes;

iii) adding H₂SO_{4,} preferably about 6 L/ton of molasses, stirring for 30 minutes and depositing for about 6 to 12 hours, evacuating the supernatant and removing sediment.

iv) diluting the supernatant and adding 1% slaked lime, stirring, boiling for 30 minutes, depositing for 7 hours and removing the black layer; and

v) conducting centrifugation to remove dirt and colloid.

In still another preferred embodiment of the process for producing high protein biomass according to the present invention, the level 3 fermentation step in the glucose medium which produced in step i) to sufficiently produce a variety of cells with a minimum cell density of 10⁸ cells/mL at temperature of 30° C., pH of 5.5, is performed under aerobic conditions with a oxygen supply from 10 L/min to 30 L/min during 16 hours, wherein:

the glucose is present in the fermentation solution at a concentration of 200 g/L, or

the glucose is in combination with molasses at a concentration of 100 g/L for glucose and 180 g/L for molasses,

and wherein:

a mineral ratio, before the fermentation, is quantified according to the glucose content as (NH₄)₂SO₄ of 25.37%; KH₂PO₄ 98% of 6.24%; MgCl₂·6H₂O of 4.23%; and CaCl₂·2H₂O of 4.23%.

In still another preferred embodiment of the process for producing high protein biomass according to the present invention, the fermentation step in aerobic conditions is carried out in the fermentation vessel selected from the group consisting of a Bioreactor fermenter and a Nalgen fermenter, with the oxygen supply of 10 L/min for the Bioreactor fermenter and 30 L/min for the Nalgen fermenter.

In still another preferred embodiment of the process for producing high protein biomass according to the present invention, the analyzing step of glucose and ethanol content in step iii), to determine the end of the fermentation to obtain the most efficient biomass, is performed in a way to optimize capacity, wherein:

for the level 1 to level 4 fermentation steps, when the remaining glucose content is from 10 to 20% of the initial glucose content, the fermentation is stopped to create a healthier strain for transculturing to commercial fermentation;

for the level 5 fermentation step, when the glucose content is from 1 to 5 g/L, the fermentation is finished to optimize capacity; and

analyzing step for ethanol content is performed to check the amount of oxygen provided during the fermentation.

According to one embodiment, the present invention is a process for producing high protein biomass from starch-containing cereal materials by yeast strains, comprising the following steps: i) producing glucose for use as a substrate from the starch-containing cereal materials by liquefaction and saccharification, including: cleaning the starch-containing cereal materials and grounding; carrying out liquefaction by raising the temperature to 85° C. and adjusting the pH to 5.5, then adding the enzyme α-amylase to carry out the liquefaction within two hours; performing saccharification by lowering the temperature to 65° C. after two hours of liquefaction, and adjusting the pH to 4.5, then quantifying the α-glucoseamylase enzyme immediately after cooling and adjusting the pH to 4.5, before the saccharification process takes place; conducting glucose content analysis by qualitative test using reagents such as iodine to determine the end of the saccharification process; and separating glucose from the residue and stores the resulting sugars; ii) selecting yeast strains which produce high protein biomass and storing the selected yeast strains, including: selecting yeast strains, which are capable of fermenting biomass, from the group consisting of S. cerevisiae, Pichia pastoris, C. utilis, Torulopsis, Geotrichum candidum, Kluyveromyces marxianus, Endomyces vernali, C. arbores, C. tropicalis, Torulopis utilis, Torulopis utilis var major, S. cerevisiae, Kluyveromyces marxianus, C. tropicalis, Monilia candia, Pichia pastoris, L. curvatus, L. reuteri, L. plantarum, L. parapentarum, and L. Pentosus; culturing on a YPG agar medium including 10 g/L of yeast extract, 20 g/L of peptone and 20 g/L of glucose at 30° C., pH of 5.5, within the range of 18 to 24 hours, to refine each strain; fermenting on the YPG agar medium including 10 g/L of yeast extract, 20 g/L of peptone and 20 g/L of glucose at 30° C., pH of 5.5, within 24 hours; sampling for glucose analysis by High Performance Liquid Chromatography (HPLC); selecting strains with the greatest biomass fermentation capacity when the residual glucose content is determined to be between 4-6 g/L; genetic sequencing to identify the yeast strains based on gene maps; and storing the selected strains; iii) producing high protein biomass from the yeast strains of step ii) by using glucose produced in step i), including: selecting yeast strains of step ii) which are propagated through three levels, level 1, level 2 and level 3, in the fermentation vessel in the corresponding medium, wherein: level 1 and level 2 fermentation are carried out in the YPG medium including 10 g/L of yeast extract, 20 g/L of peptone and 20 g/L of glucose, at 30° C. and pH of 5.5; level 3 fermentation is carried out in a medium including the glucose produced in step i) to produce a variety of cells having a minimum cell density of 108 cells/mL for the propagation step in the fermentation stage; wherein glucose is maintained at 200 g/L to optimize the fermentation; and fermenting in which this step also goes through five levels of fermentation: level 1, level 2, level 3, level 4 and level 5, with the fermentation substrate source including the glucose produced in step i) at a concentration of 200 g/L; and analyzing of glucose, sucrose and ethanol content in a fermentation solution after the fermentation process to determine the end of the process in order to obtain the most effective biomass. The starch-containing cereal materials are selected from the group consisting of cassava, maize, wheat and rice. In step i) an amount of the added α-amylase enzyme is calculated according to the starch content of 0.02% during the liquefaction. The strains with the greatest biomass fermentation capacity in step ii) are Kluyveromyces marxianus and C. utilis, with a cell density of 2.5×108 cells/mL and 5.75×108 cells/mL, respectively. Alternatively, the strain with the greatest biomass fermentation capacity in step ii) is Kluyveromyces marxianus. Alternatively, the strain with the greatest biomass fermentation capacity in step ii) is Kluyveromyces maxianus KJ830981.1. Storing of the selected strains in step ii) is carried out in an environment of 30% glycerol at a temperature of approximately −70° C. Molasses is used in combination with the glucose for fermentation, wherein the molasses is treated to de-color and remove colloid, wherein the colloid removal involves removing a colloidal system containing high viscosity protein and pectin. The colloid removal process is performed using a method selected from the group consisting of a chemical method and a mechanical method. The chemical method is performed by using H2SO4 and lime to precipitate colloid, simultaneously forming a binding with salt and decomposed protein. The mechanical method is carried out by the steps of: i) diluting the molasses with water in the ratio of molasses/water (ton/m3) of about 1.0:0.7; ii) adding CaCl2 with the ratio of CaCl2/molasses (kg/ton) of about 0.9:1, stirring for 30 minutes and depositing for 30 minutes; iii) adding H2SO4 and about 6 L/ton of molasses, stirring for 30 minutes and depositing for about 6 to 12 hours, evacuating the supernatant and removing sediment; iv) diluting the supernatant and adding 1% slaked lime, stirring, boiling for 30 minutes, depositing for 7 hours and removing the black layer; and v) conducting centrifugation to remove dirt and colloid. The level 3 fermentation step in the glucose medium which is produced in step i) to produce the variety of cells with the minimum cell density of 108 cells/mL at a temperature of 30° C., pH of 5.5, is performed under aerobic conditions with a supply of oxygen from 10 L/min to 30 L/min during 16 hours; wherein the glucose is present in the fermentation solution at a concentration of 200 g/L; and wherein a mineral ratio, before the fermentation, is quantified according to the glucose content as (NH4)2SO4 of 25.37%; KH2PO4 98% of 6.24%; MgCl2·6H2O of 4.23%; and CaCl2·2H2O of 4.23%. The fermentation step in aerobic conditions is carried out in the fermentation vessel selected from the group consisting of a Bioreactor fermenter and a Nalgen fermenter, with the oxygen supply of 10 L/min for the Bioreactor fermenter and 30 L/min for the Nalgen fermenter. The level 3 fermentation step in the glucose medium which is produced in step i) to produce the variety of cells with the minimum cell density of 108 cells/mL at a temperature of 30° C., pH of 5.5, is performed under aerobic conditions with a supply of oxygen from 10 L/min to 30 L/min during 16 hours; wherein the glucose is in combination with molasses at a concentration of 100 g/L for glucose and 180 g/L for molasses; and wherein a mineral ratio, before the fermentation, is quantified according to the glucose content as (NH4)2SO4 of 25.37%; KH2PO4 98% of 6.24%; MgCl2·6H2O of 4.23%; and CaCl2·2H2O of 4.23%. The fermentation step in aerobic conditions is carried out in the fermentation vessel selected from the group consisting of a Bioreactor fermenter and a Nalgen fermenter, with the oxygen supply of 10 L/min for the Bioreactor fermenter and 30 L/min for the Nalgen fermenter. The analyzing step of glucose and ethanol content in step iii), to determine the end of the fermentation to obtain the most efficient biomass, is performed to optimize capacity, wherein: for the level 1 to level 4 fermentation steps, when the remaining glucose content is from 10 to 20% of the initial glucose content, the fermentation is stopped to create a healthier strain for transculturing to commercial fermentation; for the level 5 fermentation step, when the glucose content is from 1 to 5 g/L, the fermentation is finished to optimize the capacity; and the analyzing step for ethanol content is performed to check the amount of oxygen provided during the fermentation.

In another embodiment, the present invention is a process for producing high protein biomass from starch-containing cereal materials by yeast strains, comprising the following steps: i) producing glucose for use as a substrate from the starch-containing cereal materials by liquefaction and saccharification, including cleaning the starch-containing cereal materials and grounding; carrying out liquefaction by raising the temperature to 85° C. and adjusting the pH to 5.5, then adding the enzyme α-amylase to carry out the liquefaction within two hours; performing saccharification by lowering the temperature to 65° C. after two hours of liquefaction, and adjusting the pH to 4.5, then quantifying the α-glucoseamylase enzyme immediately after cooling and adjusting the pH to 4.5, before the saccharification process takes place; conducting glucose content analysis by qualitative test using reagents such as iodine to determine the end of the saccharification process; and separating glucose from the residue and stores the resulting sugars; ii) selecting yeast strains which produce high protein biomass and storing the selected yeast strains, including: selecting yeast strains, which are capable of fermenting biomass, from the group consisting of S. cerevisiae, Pichia pastoris, C. utilis, Torulopsis, Geotrichum candidum, Kluyveromyces marxianus, Endomyces vernali, C. arbores, C. tropicalis, Torulopis utilis, Torulopis utilis var major, S. cerevisiae, Kluyveromyces marxianus, C. tropicalis, Monilia candia, Pichia pastoris, L. curvatus, L. reuteri, L. plantarum, L. parapentarum, and L. Pentosus; culturing on a YPG agar medium including 10 g/L of yeast extract, 20 g/L of peptone and 20 g/L of glucose at 30° C., pH of 5.5, within the range of 18 to 24 hours, to refine each strain; fermenting on the YPG agar medium including 10 g/L of yeast extract, 20 g/L of peptone and 20 g/L of glucose at 30° C., pH of 5.5, within 24 hours; sampling for glucose analysis by High Performance Liquid Chromatography (HPLC); selecting strains with the greatest biomass fermentation capacity when the residual glucose content is determined to be between 4-6 g/L; genetic sequencing to identify the yeast strains based on gene maps; and storing the selected strains; iii) producing high protein biomass from the yeast strains of step ii) by using glucose produced in step i), including: selecting yeast strains of step ii) which are propagated through three levels, level 1, level 2 and level 3, in the fermentation vessel in the corresponding medium, wherein: level 1 and level 2 fermentation are carried out in the YPG medium including 10 g/L of yeast extract, 20 g/L of peptone and 20 g/L of glucose, at 30° C. and pH of 5.5; level 3 fermentation is carried out in a medium including the glucose produced in step i) in combination with molasses, to produce a variety of cells having a minimum cell density of 108 cells/mL for the propagation step in the fermentation stage; wherein the glucose in combination with molasses is maintained at a concentration of 100 g/L for glucose and 180 g/L for molasses to optimize the fermentation; and fermenting in which this step also goes through five levels of fermentation: level 1, level 2, level 3, level 4 and level 5, with the fermentation substrate source including the glucose produced in step i) in combination with molasses at a concentration of 100 g/L for glucose and 180 g/L for molasses; and analyzing of glucose, sucrose and ethanol content in a fermentation solution after the fermentation process to determine the end of the process in order to obtain the most effective biomass. The strain with the greatest biomass fermentation capacity in step ii) is Kluyveromyces marxianus. Alternatively, the strain with the greatest biomass fermentation capacity in step ii) is Kluyveromyces maxianus KJ830981.1.

In a further embodiment, the present invention is a high protein biomass product produced from starch-containing cereal materials by a process using yeast strains, the process comprising the following steps: i) producing glucose for use as a substrate from the starch-containing cereal materials by liquefaction and saccharification, including: cleaning the starch-containing cereal materials and grounding; carrying out liquefaction by raising the temperature to 85° C. and adjusting the pH to 5.5, then adding the enzyme α-amylase to carry out the liquefaction within two hours; performing saccharification by lowering the temperature to 65° C. after two hours of liquefaction, and adjusting the pH to 4.5, then quantifying the α-glucoseamylase enzyme immediately after cooling and adjusting the pH to 4.5, before the saccharification process takes place; conducting glucose content analysis by qualitative test using reagents such as iodine to determine the end of the saccharification process; and separating glucose from the residue and stores the resulting sugars; ii) selecting yeast strains which produce high protein biomass and storing the selected yeast strains, including: selecting the yeast strains, which are capable of fermenting biomass, from the group consisting of S. cerevisiae, Pichia pastoris, C. utilis, Torulopsis, Geotrichum candidum, Kluyveromyces marxianus, Endomyces vernali, C. arbores, C. tropicalis, Torulopis utilis, Torulopis utilis var major, S. cerevisiae, Kluyveromyces marxianus, C. tropicalis, Monilia candia, Pichia pastoris, L. curvatus, L. reuteri, L. plantarum, L. parapentarum, and L. Pentosus; culturing on a YPG agar medium including 10 g/L of yeast extract, 20 g/L of peptone and 20 g/L of glucose at 30° C., pH of 5.5, within the range of 18 to 24 hours, to refine each strain; fermenting on the YPG agar medium including 10 g/L of yeast extract, 20 g/L of peptone and 20 g/L of glucose at 30° C., pH of 5.5, within 24 hours; sampling for glucose analysis by High Performance Liquid Chromatography (HPLC); selecting strains with the greatest biomass fermentation capacity when the residual glucose content is determined to be between 4-6 g/L; genetic sequencing to identify the yeast strains based on gene maps; and storing the selected strains; iii) producing high protein biomass from the yeast strains of step ii) by using glucose produced in step i), including: selecting yeast strains of step ii) which are propagated through three levels, level 1, level 2 and level 3, in the fermentation vessel in the corresponding medium, wherein: level 1 and level 2 fermentation are carried out in the YPG medium including 10 g/L of yeast extract, 20 g/L of peptone and 20 g/L of glucose, at 30° C. and pH of 5.5; level 3 fermentation is carried out in a medium including the glucose produced in step i) to produce a variety of cells having a minimum cell density of 108 cells/mL for the propagation step in the fermentation stage; wherein glucose is maintained at 200 g/L to optimize the fermentation; and fermenting in which this step also goes through five levels of fermentation: level 1, level 2, level 3, level 4 and level 5, with the fermentation substrate source including the glucose produced in step i) at a concentration of 200 g/L; and analyzing of glucose, sucrose and ethanol content in a fermentation solution after the fermentation process to determine the end of the process in order to obtain the most effective biomass, thereby producing the high protein biomass product.

### Advantageous Effects of the Present Invention

The yeast protein biomass product of the present invention is very useful in animal husbandry, especially for pig, aquatic, poultry, and large-scale animal husbandry on an industrial scale. Especially, with the control policy towards a complete ban on antibiotics in animal husbandry, products of the present invention can replace antibiotics in the livestock industry, thereby reducing imports of products from abroad.

Moreover, the process of the present invention applies high technology in the processing of starch-containing cereal materials, uses molasses as animal feed with high economic benefits, and contributes to reducing environmental pollution caused by waste of starch processing.

### BRIEF DESCRIPTION OF DRAWINGS

The foregoing summary, as well as the following detailed description of presently preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 illustrates the results of genetic sequencing of yeast strains Kluyveromyces marxianus N2.

To facilitate an understanding of the invention, identical reference numerals have been used, when appropriate, to designate the same or similar elements that are common to the figures. Further, unless stated otherwise, the features shown in the figures are not drawn to scale, but are shown for illustrative purposes only.

### DETAILED DESCRIPTION

Certain terminology is used in the following description for convenience only and is not limiting. The article “a” is intended to include one or more items, and where only one item is intended the term “one” or similar language is used. Additionally, to assist in the description of the present invention, words such as top, bottom, side, upper, lower, front, rear, inner, outer, right and left may be used to describe the accompanying figures. The terminology includes the words above specifically mentioned, derivatives thereof, and words of similar import.

Hereinafter, the present invention will be described more detail with reference to certain preferred embodiments which are only for purpose of illustrating this invention. Thus, the present invention is not only limited to these embodiments.

Apparently, the person ordinary skill in the art, based on this specification, can make modifications and/or variations that are not outside the scope of the present invention. In one embodiment, the present invention provides a process of producing high protein biomass from starch-containing cereal materials by yeast strains, with this process comprising the following step:

i) producing glucose for use as a substrate from starch-containing cereal materials by liquefaction and saccharification, including: cleaning the starch-containing cereal materials and grounding; carrying out liquefaction by raising the temperature to 85° C. and adjusting the pH to 5.5, then adding the enzyme 60 -amylase to carry out the liquefaction within two hours; performing saccharification by lowering the temperature to 65° C. after two hours of liquefaction, and adjusting the pH to 4.5, then quantifying the α-glucoseamylase enzyme immediately after cooling and adjusting the pH to 4.5, before the saccharification process takes place; conducting glucose content analysis by qualitative test using reagents such as iodine to determine the end of the saccharification process; and separating glucose from the residue and storing the resulting sugars.

Hereinafter, the above-mentioned step i) will be described more detail.

Starch-containing cereal materials are used, preferably selected from the group consisting of cassava, maize, wheat and rice, preferably cassava, finely ground, preferably up to 1 to 3 mm size, by any common grinding equipment in the art.

In addition, if necessary, starch-containing cereal materials can also be treated and cleaned before grinding. Cleaning may include, but is not limited to, washing and cleaning with water, peeling off, and rinsing several times with water.

Next, after the material is cleaned and ground to a suitable size, carrying out liquefaction is performed by adding water, raising the temperature to 85° C. and adjusting the pH of 5.5, then adding the α-amylase enzyme to carry out the liquefaction within two hours.

There are no restrictions on the water used for replenishment in the previous step, and it is possible to use domestic water, tap water, purified water, etc. The ratio of starch-containing cereal materials, preferably cassava, to the dry matter content for liquefaction and saccharification (in %) is not limited, but according to one preferred embodiment, the ratio is preferably in the range of 20 to 25% for, in the process of liquefaction and saccharification, easily stirring by a stirring shaft. If this ratio is lower than a lower limit, specifically in case of low content of dry matter, it will waste power and energy to heat up. Conversely, if this ratio is greater than an upper limit, specifically in case of very dense content of materials, stirring by the stirring shaft will be very difficult to perform.

In addition, α-amylase (EC3.2.1.3) is a hydrolytic enzyme of 1,4-α-glucoside binding in polysaccharides to produce dextrin, oligosaccharides and glucose, the amount of α-amylase enzyme added in the liquefaction when compared to the starch content is preferably in the range of 0.01 to 0.03%, more preferably around 0.02% after heating, adjusting the pH and before the liquefaction takes place. The content in this range will optimize and shorten the time of liquefaction. Conversely, if the content is lower than the lower limit, the liquefaction time is longer, and in case the content is greater than the upper limit, the cost for the enzymes is increased.

For adjusting the pH of 5.5, any common pH adjuster can be used, but according to one preferred embodiment, the pH adjuster is selected from the group consisting of a solution of H₂SO₄ and a 20% solution of NaOH.

After two hours of liquefaction, saccharizing is performed by lowering the temperature to 65° C. and adjusting the pH to 4.5 by using the pH adjuster, then quantifying the α-glucoseamylase enzyme immediately after cooling down and adjusting the pH to 4.5 before the saccharification process takes place. α-glucoseamylase is an enzyme that hydrolyses both 1,4-α-glucoside and 1,6-α-glucoside from the non-reducing end of starches, oligo-related in olysaccharides to producing glucose. A unit of activity of α-glucoseamylase (0.12% compared to starch) was determined as the amount of enzyme needed to release 1 μmol glucose per minute under experimental conditions.

Next, analyzing glucose content is performed by High Performance Liquid Chromatography (HPLC) to determine the end of the saccharification. More specifically, the end of the saccharification is the time when the glucose content is analyzed and determined to be asymptotic to a theoretical glucose content, and preferably the glucose content is about 110% compared with starch to optimize performance.

Then, at the end of the saccharification, separating glucose from the residue and storing the resulting sugars is performed. More specifically, after identifying the end of the saccharification, a screw-shaft pump on a pulp separator is used with a horizontal-shaft centrifugation having an external filter and a helical screw-shaft to extrude the pulp at the end of helical screw-shaft to increase pulp separation efficiency. Separated sludge is stored in silos for being transferred directly to the animal feeding system. The filtrate contains glucose (200 g/L), then this filtrate will go through a sand separation stage by specialized equipment, then transported to a tank for the next fermentation.

It should be noted that starch-containing cereal materials according to the present invention are very readily available and the most preferred is cassava because of lower cost while the starch yield of cassava is higher than other cereals. However, in another embodiment, other starch-containing cereal materials include, but not limited to, corn, wheat and rice.

The use of these materials is similar to that of cassava, firstly saccharizing the starch-containing materials into glucose, then using glucose to ferment for producing yeast biomass.

Normally, 1 ton of starch produces 1.1 tons of glucose. In order to produce 1 ton of biomass (54.6% protein) requires 1.96 tons of glucose (Gustavo Graciano Fonseca, May 2007), or 1.781 tons of starch respectively, equivalent to cassava/maize/wheat/rice of respectively 2.8/2.3/2.4/1.9 tons.

Therefore, based on this description, the person ordinary skill in the art can easily make the present invention with the other materials equivalent to those described above.

The process of producing high protein biomass from starch-containing cereal materials by yeast strains further comprises step ii) as described below:

ii) selecting yeast strains which produce high protein biomass and storing the selected yeast strains, including:

selecting the yeast strains, which are capable of fermenting biomass, include S. cerevisiae, Pichia pastoris, C. utilis, Torulopsis, Geotrichum candidum, Kluyveromyces marxianus, Endomyces vernali, C. arbores, C. tropicalis, Torulopis utilis, Torulopis utilis var major, S. cerevisiae, Kluyveromyces marxianus, C. tropicalis, Monilia candia, Pichia pastoris, L. curvatus, L. reuteri, L. plantarum, L. parapentarum, and L. Pentosus;

culturing on a YPG agar medium including 10 g/L of yeast extract, 20 g/L of peptone and 20 g/L of glucose at 30° C., pH of 5.5, within the range of 18 to 24 hours, to refine each strain;

fermenting on the YPG agar medium including 10 g/L of yeast extract, 20 g/L of peptone and 20 g/L of glucose at 30° C., pH of 5.5, within 24 hours;

sampling for glucose analysis by High Performance Liquid Chromatography (HPLC);

selecting strains, preferably one or two strains, with the greatest biomass fermentation capacity when the residual glucose content is determined to be between 4-6 g/L;

genetic sequencing to identify yeast strains based on gene maps; and

storing or otherwise preserving the selected strains.

Hereinafter, the above-mentioned step ii) will be described more detail.

The strains of yeast were first collected. More specifically, the inventors use ten strains of yeast from the Institute of Biotechnology-Vietnam Academy of Sciences and ten strains of yeast from the Department of Biotechnology, University of Chemistry and Technology, Prague, Czech Republic to ferment biomass in the Bioreactor, to find yeast strains capable of fermenting biomass.

In one specific preferred embodiment, these strains include:

ten strains of yeast from the Department of Biotechnology, University of Chemistry and Technology, Prague-Czech Republic: S. cerevisiae, Pichia pastoris, C. utilis, Torulopsis, Geotrichum candidum, Kluyveromyces marxianus, Endomyces vernali, C. arbores, C. tropicalis, Torulopis utilis, and Torulopis utilis var major; and

ten strains of yeast from the Institute of Biotechnology-Vietnam Academy of Sciences: S. cerevisiae, Kluyveromyces marxianus, C. tropicalis, Monilia candia, Pichia pastoris, L. curvatus, L. reuteri, L. plantarum, L. parapentarum, and L. Pentosus.

It should be noted that these strains were selected because previous studies have demonstrated that these strains are capable of fermenting biomass. However, the present invention is not limited to the above-selected specific strains, but other suitable biomass fermentation strains can all be used according to the present invention.

Then, the process includes culturing on a YPG agar medium to refine each strain, and after that, fermenting on the YPG agar medium including 10 g/L of yeast extract, 20 g/L of peptone and 20 g/L of glucose, at 30° C., pH of 5.5, within the range of 18 to 24 hours.

In this step, from the sixth hour onwards, samples are taken (one sample every two hours) to check parameters such as contaminants, and to analyze sugar content by liquid High Performance Liquid Chromatography (HPLC), such that when the remaining glucose content is from 4 to 6 g/L, the process ends.

From the results, two strains, Kluyveromyces marxianus and Candida utilis, are the two strains of yeast with the highest capacity of producing biomass. Specifically, the capacity of Kluyveromyces marxianus (or K. Marxianus) is 2.5×10⁸ cells mL and the capacity of Candida utilis (or C. utilis) is 5.75×10⁸ cells/mL. The strains of Kluyveromyces marxianus are preferred to ferment biomass and conduct genetic sequencing because they are safer, while Candida utilis is related to some fungi that cause skin diseases in humans.

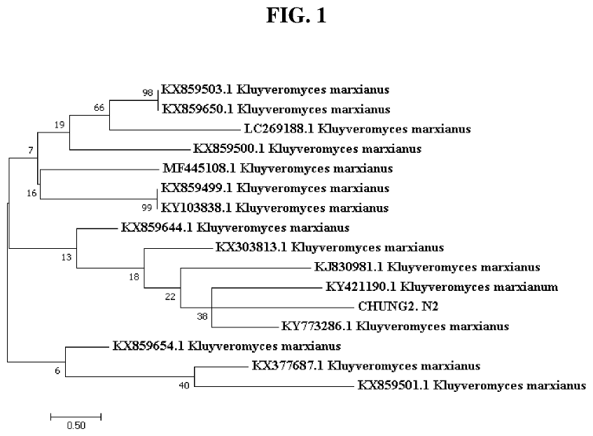

The result of genetic sequencing is as shown in FIG. 1.

As shown in FIG. 1, from a plant derived from the strain of the yeast strain Kluyveromyces marxianus N2, it could be seen that the yeast strain N2 is closely related to the strain Kluyveromyces marxianus (KJ83098.1) with the sequence homology is 99%, and the coverage is 100%.

After selecting the strains, the process stores them in 30% glycerol at a temperature of about −70° C.

The process of producing high protein biomass from starch-containing cereal materials by yeast strains further comprises step iii) as described below:

iii) producing high protein biomass from yeast strains of step ii) by using glucose produced in step i), wherein:

the selected yeast strains of step ii) are propagated through three levels, level 1, level 2 and level 3, in the fermentation vessel in the corresponding medium, wherein:

level 1 and level 2 fermentation are carried out in the YPG medium including 10 g/L of yeast extract, 20 g/L of peptone and 20 g/L of glucose, at 30° C. and pH of 5.5;

level 3 fermentation is carried out in a medium selected from the group consisting of: glucose produced in step i) and glucose produced in step i) in combination with molasses, to sufficiently produce a variety of cells having a minimum cell density of 10⁸ cells/mL for the propagation step in the fermentation stage on the industrial scale, wherein:

glucose is maintained at 200 g/L, or glucose in combination with molasses at a concentration of 100 g/L for glucose and 180 g/L for molasses to optimize the fermentation is maintained;

fermenting on an industrial scale, in which this step also goes through five levels of fermentation, level 1, level 2, level 3, level 4 and level 5, with the fermentation substrate source selected from the group of glucose produced in step i) at a concentration of 200 g/L and glucose produced in step i) in combination with molasses at a concentration of 100 g/L for glucose and 180 g/L for molasses; and

analyzing of glucose, sucrose and ethanol content in the fermentation solution after fermentation process to determine the end of the process in order to obtain the most effective biomass.

Hereinafter, the above-mentioned step iii) will be described more detail.

The glucose produced in step i) and the yeast strains selected in step ii) will be used to produce high protein biomass.

First, the yeast strains selected in step ii) are propagated through three levels, level 1, level 2 and level 3, in the fermentation vessel in the corresponding medium, wherein:

level 1 and level 2 fermentation are carried out in a YPG medium including 10 g/L of yeast extract, 20 g/L of peptone and 20 g/L of glucose, at 30° C. and pH of 5.5;

level 3 fermentation is carried out in a medium selected from the group consisting of: glucose produced in step i) and glucose produced in step i) in combination with molasses, to sufficiently produce a variety of cells having cell density at least of 10⁸ cells/mL for the propagation step in the fermentation stage on the industrial scale, such that the temperature of this fermentation step is 30° C., the pH is 5.5 and the fermentation is carried out under aerobic conditions with the oxygen supply from 10 L/min to 30 L/min in 16 hours.

For fermentation under aerobic conditions, the amount of supplied oxygen is important because it is closely related to the growth and development of yeast. More specifically, to optimize this parameter, the inventors studied to determine oxygen demand, based on the oxygen content in yeast cells of 28%. The calculation of oxygen demand is shown in Table 5 below.

TABLE 5
THE CALCULATION OF OXYGEN DEMAND

| | Bioreactor | Nalgen |
|---|---|---|
| K, amount of fermentation (L) | 4 | 10 |
| Glucose content (g/L) | 200 | 200 |
| K, amount of glucose/fermentation batch (g) | 800 | 2000 |
| Estimated biomass production is 51% | 408 | 1.020 |
| compared to glucose | | |
| Oxygen demand (L) | 0.11 | 0.29 |
| corresponding air (L) | 0.71 | 1.79 |
| Ability to dissolve air in water | 0.01% | 0.01% |
| Total amount of air supply for 10 hours of | 7,140 | 17,850 |
| fermentation (L) | | |
| Air supply per minute | 11.9 | 29.75 |

For optimizing the fermentation, the glucose content is maintained at a concentration of 200 g/L, or the glucose in combination with molasses is maintained at a concentration of 100 g/L for glucose and 180 g/L for molasses, and wherein the mineral ratio, before performing the fermentation, is quantified according to the glucose content as (NH₄)₂SO₄ of 25.37%; KH₂PO₄ 98% of 6.24%; MgCl₂·6H₂O of 4.23%; and CaCl₂·2H₂O of 4.23%.

The basis for calculating mineral contents depend on the ingredients of biomass (nitrogen: 8-10%; phosphorus: 3%; potassium 0%; Mg 2%; Ca 3%), using (NH₄)₂SO₄ as a source of nitrogen; KH₂PO₄ 98% as source of phosphorus and potassium; MgCl₂·6H₂O as a source of Mg; and CaCl₂·2H₂O as a source of calcium. Based on the above required mineral contents in biomass, the salt amounts are calculated in which the demand of salts as (NH₄)₂SO₄ of 26%; KH₂PO₄ 98%; MgCl₂·6H₂O and CaCl₂·2H₂O at 50% of the demand amount because Mg and Ca are available at 50% in water.

The fermentation is then carried out on an industrial scale, in which this step also goes through five levels of fermentation, level 1, level 2, level 3, level 4 and level 5, with the fermentation substrate source selected from the group of glucose produced in step i) at a concentration of 200 g/L and glucose produced in step i) in combination with molasses at a concentration of 100 g/L for glucose and 180 g/L for molasses.

Finally, analyzing of glucose, sucrose and ethanol content is performed in the fermentation solution after fermentation to determine the end of the process in order to obtain the most effective biomass.

As described above, the purpose of analyzing the content of glucose and ethanol is to determine the end of the fermentation, thereby obtaining the most efficient biomass. For level 1 to level 4 fermentations, when the remaining glucose content is about 10 to 20% of the initial glucose content, the fermentation is completed, in order to create a healthier strain seed for inoculating to fermentation in commercial products. In addition, for the final fermentation, the glucose content is preferably in the range of 1 to 5 g/L because the yeast biomass decreases due to the cytoplasm of the yeast being consumed, as well as due to extending the time which reduces the capacity of the industrial plant. On the contrary, if the fermentation time is too short, it will consume yeast biomass because it does not make full use of the cytoplasm of yeast.

In addition, the ethanol test is to determine if the fermentation has enough oxygen, because in the absence of oxygen, the synthesis of proteins will turn to the ethanol-producing fermentation, and then the fermentation to converting ethanol to protein takes more time, thus the capacity of the industrial plant is reduced due to the extended time.

Moreover, as described above, glucose is the suitable substrate for fermentation. However, in another embodiment, the glucose produced in the above step i) can be combined with molasses. Molasses is also a good substrate for fermenting microorganisms to create valuable products and, if used, molasses should be treated to de-color and remove colloid.

It is necessary to de-color because the color of molasses is difficult to destroy, but easily attaches to the yeast biomass and causes a dark yellow color for the yeast, and this will affect the color and quality of yeast biomass. There are no specific limits for a method for de-coloring of molasses and therefore any common method for de-coloring can be used, for example, by de-coloring with activated carbon.

The colloid present in molasses is a colloidal system consisting of high viscosity protein and pectin that reduces the solubility of oxygen, which hinders the metabolism of fungal cells, so this colloidal system needs to be destroyed in order to reduce cell influence, and increase the efficiency of yeast biomass acquisition.

For destroying colloid, any common method in the art for destroying colloid can be used; however, in one preferred embodiment, the method for destroying colloid is selected from the group consisting of a chemical method and a mechanical method.

The chemical method is performed by using H₂SO₄ and lime to precipitate colloid, and to simultaneously form a binding with salt and decomposed protein.

The mechanical method is carried out by the following steps:

i) diluting molasses with water in the ratio of molasses/water (ton/m³) of about 1.0:0.7;

ii) adding CaCl₂ with the ratio of CaCl₂/molasses (kg/ton) of about 0.9:1, stirring for 30 minutes and depositing for 30 minutes;

iii) adding H₂SO₄, preferably about 6 L/ton of molasses, stirring for 30 minutes and depositing for about 6 to 12 hours, evacuating the supernatant and removing sediment.

iv) diluting the supernatant and adding 1% slaked lime, stirring, boiling for 30 minutes, depositing for 7 hours and removing the black layer; and

v) conducting centrifugation to remove dirt and colloid.

In addition, any equipment commonly used for aerobic fermentation in the art can be used to carry out the fermentation, but in one preferred embodiment, the fermentation vessel is preferably selected from the group consisting of a Bioreactor fermenter and a Nalgen fermenter, with the oxygen supply of 10 L/min for the Bioreactor fermenter and 30 L/min for the Nalgen fermenter.

Bioreactor is a fermenter that can automatically perform the fermentation, adjust pH, prevent foam, and adjust the amount of air injected. In short, this device is a special-purpose one for fermenting because all the fermentation is carried out according to a program pre-set with the fermentation parameters in the operating software, and the parameters are recorded by itself. During the fermentation, the device draws and/or displays a diagram or chart of parameters over time.

In addition, Nalgen is a type of large capacity fermenter, having a cap which has drilled holes to install valves and air pipes, seed pumps, and an autosampler. Although the whole fermentation is not automatic, it has an ability to ferment in a way that is larger than the Bioreactor.

It should be understood that the above description is only for illustrative purposes and therefore does not limit the present invention in any way.

Apparently, the person ordinary skill in the art, based on this specification, can make modifications and/or variations that are corresponded with the specifically embodiments of the present invention.

### EXAMPLES

Hereinafter, the present invention will be described in more detail through examples which are only for illustrative purposes. It should be noted that the steps, components and conditions are preferably, but are not limited to, the steps, components, and conditions as specifically described above and below.

### Example 1

### Researching Gelatinization, Liquefaction and Saccharification of the Starch-containing Cereal Materials

The gelatinization, liquefaction and saccharification were carried out according to Vu V H et al. 2009, with improvements to optimize in accordance with actual conditions.

Starch-containing cereal materials: the material is cassava that is washed and ground to a particle size of 1-3 mm.

Study on liquefaction and saccharification: three empiric formulae are prepared with a ratio of cassava and water being, respectively, ⅕, ¼ and ⅓;

21 empiric formulae for liquefaction and saccharification are prepared, wherein:

for empiric formula 1: the ratio of cassava/water=⅕;

for empiric formula 2: the ratio of cassava/water=¼;

for empiric formula 3: the ratio of cassava/water=⅓; and in which:

each experiment was divided into seven different empiric formulae and repeated three times for each empiric formula, and the total number of experiments was 63.

It should be noted that, during implementation, a sample will be taken for the glucose content analysis and the qualitative glucose test with iodine will be performed to determine the end of the saccharification as described herein.

Experimental Layout

a) 16 g of cassava+100 mL of water were added into a 500 mL flask;

b) 16 g of cassava+100 mL of water+2μL of α-amylase enzyme were added into a 500 mL flask;

c) 16 g of cassava+100 mL of water were added into a 500 mL flask;

d) 16 g of cassava+100 mL of water+5μL of α-amylase enzyme were added into a 500 mL flask.

The four flasks a)-c) above are placed at temperatures of respectively 65° C., 70° C., 75° C. and 80° C. for 30 mins, then used to carry out the next step as following:

Take flasks a) and c) and add 20 μL of α-amylase enzyme (two flasks (e)).

The flasks in the above steps were placed at temperatures of 65° C., 70° C., 75° C. and 80° C. for 60 minutes, then proceed to add the San Extra enzyme as follows:

Flasks b) and e) are added with α-amylase in a ratio of 5/10/15 μL α-amylase enzyme, respectively.

Flask a) is supplemented with α-amylase in a ratio of 5/10/15 μL α-amylase enzyme, respectively.

From the above experiments, the degree of the saccharification, the time of the saccharification, and enzyme content, etc., were determined as described above in order to optimize gelatinization, liquefaction and saccharification of the starch-containing cereal materials.

### Example 2

### Separation of Glucose from the Pulp

After conducting a glucose content analysis by qualitative analysis using reagents such as iodine as described above, the end of the above saccharification was determined, and the glucose was separated and stored in order to use for the next step.

Glucose can be separated by suitable common methods in the art. However, in this embodiment, glucose is separated from the pulp by any method selected in the group of, but is not limited to, centrifugation, fin filter and press frame.

For the person ordinary skill in the art, these above methods are common and widely used, so a more detailed description of this method is considered unnecessary.

### Example 3

### Selection Study and Selection of Yeast Strains that Highly Produce Protein and Optimizing Conditions of the Fermentation for Producing Biomass of Seed

### Example 3.1

### Collection and Activation of Yeast Strains

Collection of yeast strains: collect twenty yeast strains from the strain bank of the Institute of Biotechnology-Vietnam Academy of Sciences and the Department of Biotechnology, University of Chemistry and Technology, Prague, Czech Republic.

More specifically, the strains were collected as following: S. cerevisiae, Pichia pastoris, C. utilis, Torulopsis, Geotrichum candidum, Kluyveromyces marxianus. Endomyces vernali, C. arbores, C. tropicalis, Torulopis utilis, Torulopis utilis var major, S. cerevisiae, Kluyveromyces marxianus, C. tropicalis; Monilia candia, Pichia pastoris, L. curvatus, L. reuteri, L. plantarum, L. Parapentarum và L. Pentosus.

The inventors used preliminary results of studies of these entities on the above strains to continue screening and selecting highly protein-productive microorganisms. These strains have had initial evaluation studies on their ability to produce proteins. Highly protein-productive biosynthetic strains are fermented on commercially available media.

Activation of the Yeast Strains

All of the strains, which have the capability of fermenting biomass, are activated by culturing on agar plates and isolated until each reaches the purity as described above before being inoculated in the fermentation solution, substantially to perform cell count and biomass quantification.

The above twenty strains were used, each strain was activated on three plates, and the total number of cultural agar plates was sixty.

### Example 3.2

### Culture in Flasks

After refining the strains, each of these strains is cultured in a basic YPD solution in a flask, at 30° C. for a period of 18 to 24 hours.

As shown above, twenty strains were isolated and cultured, each of which was then fermented three times.

The conditions to be assessed include heat, stirring, fermentation, etc., in order to optimize the fermentation conditions (as shown above). In addition, the ratio and ingredients of the culture medium are also optimized by the multivariate-response surface method (RSM) according to Vu V H, Kim K. 2009, Kim et al. 2007 with improvements.

The multivariate-response surface method is specifically described in the prior art, so a more detailed description of this method is considered unnecessary.

### Example 3.3

### Test of Yeast Morphology, Enumeration of Cells, Yeast Biomass, Selection of Yeast Strains

The process determines the number of cells and the cell biomass content. After fermentation, the sample in each flask will be taken out and checked on a microscope to count the cells. The strains with the highest cell density are continuously fermented to select from one to two strains with the highest biomass fermentation capacity (evaluated by Branford, 1976 with improvements).

Yeast biomass was determined. The strains with high protein-producing synthesis meet the industry's objectives and standards, include characteristics that are easy to raise in industrial environments, adaptable over a wide temperature range, high protein-producing capacity, and stable, and the strains must be healthy in terms of growth and development.

These selected strains were classified according to a molecular biology method.

As a result, the strains with the highest biomass fermentation capacity were Kluyveromyces marxianus and C. utilis, with the cell densities of 2.5×10⁸ cells/mL and 5.75×10⁸ cells/mL, respectively.

Kluyveromyces marxianus is preferred to ferment biomass and conduct genetic sequencing because they are safer, while Candida utilis is related to some fungi that cause skin diseases in humans.

### Example 3.4

### Genetic Sequencing

Identifying the Selected Strains

The selected strains were extracted from total DNA, and cloned using ITS 16S by polymerase chain reaction (PCR). Post-purified PCR products are sent to the Macrogen Center in Korea or the National Key Laboratory of Genomics, Institute of Biotechnology, Vietnam Academy of Science and Technology for sequencing.

The genealogy analysis was then performed to determine the genetic relationship with other bacterial strains according to Sambrook & Russell (2001) (J., D., 2001), White et al. 1990.

Next, some biological characteristics are evaluated, which include the ability to use carbon sources according to BioMérieux's API® KIT, nitrogen sources, antibiotic resistance, etc., of selected strains on fermentation conditions and ingredients of a medium according to the thesis of Dr. Vu V H, 2009.

The results of the genetic sequencing show that the strain with the most biomass fermentation capacity is Kluyveromyces marxianus KJ830981.1.

### Example 3.5

### Storing the Selected Strains

The conditions for storing or otherwise preserving the selected strains were investigated according to the strain preservation method described in Alexander G A. 1981; Ryan Dwet al. 2010 with improvements, at −86° C. and −20° C. to determine optimal storage conditions. More specifically, the strains are stored or otherwise preserved in glycerol of 10-40%, at +5° C. to −86° C. This process was carried out for 12 months, in which cultured cells were taken out each month for counting to assess survival, and each experiment was repeated three times; or when the ratio of sucrose is 10-40%, at room temperature. Similarly, this process was carried out for 12 months, in which cultured cells were taken out each month for counting to assess survival, and each experiment was repeated three times.

The results show that, although the above storage conditions are applicable to the present invention, it is the best to store the selected strains in 30% glycerol at a temperature of about −70° C. in terms of survival possibility of the stored or preserved strains.

### Example 4

### The Process of High Protein Biomass Production by Yeast

### Example 4.1

### Liquefaction and Saccharification of Cassava

Performing the procedure shown above and in Example 1 produce glucose for fermentation in this example. 500 kg of cassava and any necessary chemicals to perform the liquefaction and saccharification of cassava were prepared to adjust the pH to 5.5 during the liquefaction and saccharification, and simultaneously using α-amylase enzyme to perform saccharification.

### Example 4.2

### High Protein Biomass Production from Yeast Strains of Example 3 Above

The strains of yeast selected in Example 3 are propagated through three levels, level 1, level 2 and level 3, in the fermentation vessel in the corresponding medium before being propagated at the fermentation stage along industrial lines.

For level 1 and level 2 fermentation, these processes are carried out in a YPG medium including 10 g/L of yeast extract, 20 g/L of peptone and 20 g/L of glucose, at 30° C. and pH of 5.5.

For level 3 fermentation, a medium is selected from the group consisting of: glucose produced in Example 1, with concentration of 200 g/L, and glucose produced in Example 1 in combination with molasses, and preferably the molasses has been treated to de-color and remove colloid as described above, at a concentration of 100 g/L for glucose and 180 g/L for molasses, to sufficiently produce a variety of cells having a minimum cell density of 10⁸ cells/mL. This fermentation is carried out at a temperature of 30° C., pH of 5.5, under aerobic conditions with the oxygen supply from 10 L/min to 30 L/min in 16 hours, wherein the mineral ratio, before performing the fermentation, is quantified according to the glucose content as (NH₄)₂SO₄ of 25.37%; KH₂PO₄ 98% of 6.24%; MgCl₂·6H₂O of 4.23%; and CaCl₂·2H₂O of 4.23%.

After that, the fermented strain was fermented on an industrial scale, in which this fermentation went through five levels of fermentation, level 1, level 2, level 3, level 4 and level 5, with the fermentation substrate source selected from the group of glucose in Example 1 at a concentration of 200 g/L and glucose in Example 1 in combination with molasses at a concentration of 100 g/L for glucose and 180 g/L for molasses.

### Example 5

### Optimization of High Protein Biomass Production by Yeast

For optimizing the capacity, the contents of glucose, sucrose and ethanol in the fermentation solution after fermentation were analyzed to determine the end of the process in order to obtain the most effective biomass.

Specifically, for level 1 to level 4 fermentations, when the remaining glucose content is about 10 to 20% of the initial glucose content, the fermentation is completed, in order to create a healthier strain seed for inoculating to fermentation in commercial products. In addition, for the final fermentation, the glucose content is preferably in the range of 1 to 5 g/L because the yeast biomass decreases due to the cytoplasm of the yeast being consumed, as well as extending the time which will reduce the capacity of the industrial plant. On the contrary, if the fermentation time is too short, it will consume yeast biomass because it does not make full use of the cytoplasm of yeast.

In addition, the ethanol test is performed to determine if the fermentation has enough oxygen, because in the absence of oxygen, the synthesis of proteins will turn to the ethanol-producing fermentation, and then the fermentation to convert ethanol to protein takes more time, thus the capacity of the industrial plant is reduced due to the extended time.

### ABILITY OF INDUSTRIAL APPLICATION

The present invention provides a process for producing high protein biomass from starch-containing cereal materials by yeast strains. The yeast-based protein product of the present invention is useful as animal feed with high economic benefits in the animal husbandry industry, such as raising pigs, aquatic products, poultry and cattle on an industrial scale, and simultaneously to contribute to reducing environmental pollution due to starch processing waste.

The present invention allows building a modern, industrial-scale fermentation technology line with the desire to contribute proactively to protein materials and some biological active ingredients used in the production of animal feed.

The present invention may be embodied in other specific forms without departing from its spirit or essential characteristics. The described embodiments are to be considered in all respects only as illustrative and not restrictive. The scope of the invention, therefore, will be indicated by claims rather than by the foregoing description. All changes, which come within the meaning and range of equivalency of the claims, are to be embraced within their scope.

What is claimed is:

1. A process for producing high protein biomass from starch-containing cereal materials by yeast strains, comprising the following steps:
i) producing glucose for use as a substrate from the starch-containing cereal materials by liquefaction and saccharification, including:
cleaning the starch-containing cereal materials and grounding;
carrying out liquefaction by raising the temperature to 85° C. and adjusting the pH to 5.5, then adding the enzyme α-amylase to carry out the liquefaction within two hours;
performing saccharification by lowering the temperature to 65° C. after two hours of liquefaction, and adjusting the pH to 4.5, then quantifying the α-glucoseamylase enzyme immediately after cooling and adjusting the pH to 4.5, before the saccharification process takes place;
conducting glucose content analysis by qualitative test using reagents such as iodine to determine the end of the saccharification process; and
separating glucose from the residue and stores the resulting sugars;
ii) selecting yeast strains which produce high protein biomass and storing the selected yeast strains, including:
selecting yeast strains, which are capable of fermenting biomass, from the group consisting of S. cerevisiae, Pichia pastoris, C. utilis, Torulopsis, Geotrichum candidum, Kluyveromyces marxianus, Endomyces vernali, C. arbores, C. tropicalis, Torulopis utilis, Torulopis utilis var major, S. cerevisiae, Kluyveromyces marxianus, C. tropicalis, Monilia candia, Pichia pastoris, L. curvatus, L. reuteri, L. plantarum, L. parapentarum, and L. Pentosus;
culturing on a YPG agar medium including 10 g/L of yeast extract, 20 g/L of peptone and 20 g/L of glucose at 30° C., pH of 5.5, within the range of 18 to 24 hours, to refine each strain;
fermenting on the YPG agar medium including 10 g/L of yeast extract, 20 g/L of peptone and 20 g/L of glucose at 30° C., pH of 5.5, within 24 hours;
sampling for glucose analysis by High Performance Liquid Chromatography (HPLC);
selecting strains with the greatest biomass fermentation capacity when the residual glucose content is determined to be between 4-6 g/L;
genetic sequencing to identify the yeast strains based on gene maps; and
storing the selected strains;
iii) producing high protein biomass from the yeast strains of step ii) by using glucose produced in step i), including:
selecting yeast strains of step ii) which are propagated through three levels, level 1, level 2 and level 3, in the fermentation vessel in the corresponding medium, wherein:
level 1 and level 2 fermentation are carried out in the YPG medium including 10 g/L of yeast extract, 20 g/L of peptone and 20 g/L of glucose, at 30° C. and pH of 5.5;
level 3 fermentation is carried out in a medium including the glucose produced in step i) to produce a variety of cells having a minimum cell density of 10⁸ cells/mL for the propagation step in the fermentation stage;
wherein glucose is maintained at 200 g/L to optimize the fermentation; and
fermenting in which this step also goes through five levels of fermentation: level 1, level 2, level 3, level 4 and level 5, with the fermentation substrate source including the glucose produced in step i) at a concentration of 200 g/L; and
analyzing of glucose, sucrose and ethanol content in a fermentation solution after the fermentation process to determine the end of the process in order to obtain the most effective biomass.

2. The process according to claim 1, wherein the starch-containing cereal materials are selected from the group consisting of cassava, maize, wheat and rice.

3. The process according to claim 1, wherein in step i) an amount of the added α-amylase enzyme is calculated according to the starch content of 0.02% during the liquefaction.

4. The process according to claim 1, wherein the strains with the greatest biomass fermentation capacity in step ii) are Kluyveromyces marxianus and C. utilis, with a cell density of 2.5×10⁸ cells/mL and 5.75×10⁸ cells/mL, respectively.

5. The process according to claim 4, wherein the strain with the greatest biomass fermentation capacity in step ii) is Kluyveromyces marxianus.

6. The process according to claim 4, wherein the strain with the greatest biomass fermentation capacity in step ii) is Kluyveromyces maxianus KJ830981.1.

7. The process according to claim 1, wherein storing of the selected strains in step ii) is carried out in an environment of 30% glycerol at a temperature of approximately −70° C.

8. The process according to claim 1, wherein molasses is used in combination with the glucose for fermentation, wherein the molasses is treated to de-color and remove colloid, wherein the colloid removal involves removing a colloidal system containing high viscosity protein and pectin.

9. The process according to claim 8, wherein the colloid removal process is performed using a method selected from the group consisting of a chemical method and a mechanical method.

10. The process according to claim 9, wherein the chemical method is performed by using H₂SO₄ and lime to precipitate colloid, simultaneously forming a binding with salt and decomposed protein.

11. The process according to claim 9, wherein the mechanical method is carried out by the steps of:
i) diluting the molasses with water in the ratio of molasses/water (ton/m³) of about 1.0:0.7;
ii) adding CaCl₂ with the ratio of CaCl₂/molasses (kg/ton) of about 0.9:1, stirring for 30 minutes and depositing for 30 minutes;
iii) adding H₂SO₄ and about 6 L/ton of molasses, stirring for 30 minutes and depositing for about 6 to 12 hours, evacuating the supernatant and removing sediment;
iv) diluting the supernatant and adding 1% slaked lime, stirring, boiling for 30 minutes, depositing for 7 hours and removing the black layer; and
v) conducting centrifugation to remove dirt and colloid.

12. The process according to claim 1, wherein the level 3 fermentation step in the glucose medium which is produced in step i) to produce the variety of cells with the minimum cell density of 10⁸ cells/mL at a temperature of 30° C., pH of 5.5, is performed under aerobic conditions with a supply of oxygen from 10 L/min to 30 L/min during 16 hours;
wherein the glucose is present in the fermentation solution at a concentration of 200 g/L; and
wherein a mineral ratio, before the fermentation, is quantified according to the glucose content as (NH₄)₂SO₄ of 25.37%; KH₂PO₄ 98% of 6.24%; MgCl₂·6H₂O of 4.23%; and CaCl₂·2H₂O of 4.23%.

13. The process according to claim 12, wherein the fermentation step in aerobic conditions is carried out in the fermentation vessel selected from the group consisting of a Bioreactor fermenter and a Nalgen fermenter, with the oxygen supply of 10 L/min for the Bioreactor fermenter and 30 L/min for the Nalgen fermenter.

14. The process according to claim 1, wherein the level 3 fermentation step in the glucose medium which is produced in step i) to produce the variety of cells with the minimum cell density of 10⁸ cells/mL at a temperature of 30° C., pH of 5.5, is performed under aerobic conditions with a supply of oxygen from 10 L/min to 30 L/min during 16 hours;
wherein the glucose is in combination with molasses at a concentration of 100 g/L for glucose and 180 g/L for molasses; and
wherein a mineral ratio, before the fermentation, is quantified according to the glucose content as (NH₄)₂SO₄ of 25.37%; KH₂PO₄ 98% of 6.24%; MgCl₂·6H₂O of 4.23%; and CaCl₂·2H₂O of 4.23%.

15. The process according to claim 14, wherein the fermentation step in aerobic conditions is carried out in the fermentation vessel selected from the group consisting of a Bioreactor fermenter and a Nalgen fermenter, with the oxygen supply of 10 L/min for the Bioreactor fermenter and 30 L/min for the Nalgen fermenter.

16. The process according to claim 1, wherein the analyzing step of glucose and ethanol content in step iii), to determine the end of the fermentation to obtain the most efficient biomass, is performed to optimize capacity, wherein:
for the level 1 to level 4 fermentation steps, when the remaining glucose content is from 10 to 20% of the initial glucose content, the fermentation is stopped to create a healthier strain for transculturing to commercial fermentation;
for the level 5 fermentation step, when the glucose content is from 1 to 5 g/L, the fermentation is finished to optimize the capacity; and
the analyzing step for ethanol content is performed to check the amount of oxygen provided during the fermentation.

17. A process for producing high protein biomass from starch-containing cereal materials by yeast strains, comprising the following steps:
i) producing glucose for use as a substrate from the starch-containing cereal materials by liquefaction and saccharification, including:
cleaning the starch-containing cereal materials and grounding;
carrying out liquefaction by raising the temperature to 85° C. and adjusting the pH to 5.5, then adding the enzyme α-amylase to carry out the liquefaction within two hours;
performing saccharification by lowering the temperature to 65° C. after two hours of liquefaction, and adjusting the pH to 4.5, then quantifying the α-glucoseamylase enzyme immediately after cooling and adjusting the pH to 4.5, before the saccharification process takes place;
conducting glucose content analysis by qualitative test using reagents such as iodine to determine the end of the saccharification process; and
separating glucose from the residue and stores the resulting sugars;
ii) selecting yeast strains which produce high protein biomass and storing the selected yeast strains, including:
selecting yeast strains, which are capable of fermenting biomass, from the group consisting of S. cerevisiae, Pichia pastoris, C. utilis, Torulopsis, Geotrichum candidum, Kluyveromyces marxianus, Endomyces vernali, C. arbores, C. tropicalis, Torulopis utilis, Torulopis utilis var major, S. cerevisiae, Kluyveromyces marxianus, C. tropicalis, Monilia candia, Pichia pastoris, L. curvatus, L. reuteri, L. plantarum, L. parapentarum, and L. Pentosus;
culturing on a YPG agar medium including 10 g/L of yeast extract, 20 g/L of peptone and 20 g/L of glucose at 30° C., pH of 5.5, within the range of 18 to 24 hours, to refine each strain;
fermenting on the YPG agar medium including 10 g/L of yeast extract, 20 g/L of peptone and 20 g/L of glucose at 30° C., pH of 5.5, within 24 hours;
sampling for glucose analysis by High Performance Liquid Chromatography (HPLC);
selecting strains with the greatest biomass fermentation capacity when the residual glucose content is determined to be between 4-6 g/L;
genetic sequencing to identify the yeast strains based on gene maps; and
storing the selected strains;
iii) producing high protein biomass from the yeast strains of step ii) by using glucose produced in step i), including:
selecting yeast strains of step ii) which are propagated through three levels, level 1, level 2 and level 3, in the fermentation vessel in the corresponding medium, wherein:
level 1 and level 2 fermentation are carried out in the YPG medium including 10 g/L of yeast extract, 20 g/L of peptone and 20 g/L of glucose, at 30° C. and pH of 5.5;
level 3 fermentation is carried out in a medium including the glucose produced in step i) in combination with molasses, to produce a variety of cells having a minimum cell density of 10⁸ cells/mL for the propagation step in the fermentation stage;
wherein the glucose in combination with molasses is maintained at a concentration of 100 g/L for glucose and 180 g/L for molasses to optimize the fermentation; and
fermenting in which this step also goes through five levels of fermentation: level 1, level 2, level 3, level 4 and level 5, with the fermentation substrate source including the glucose produced in step i) in combination with molasses at a concentration of 100 g/L for glucose and 180 g/L for molasses; and
analyzing of glucose, sucrose and ethanol content in a fermentation solution after the fermentation process to determine the end of the process in order to obtain the most effective biomass.

18. The process according to claim 17, wherein the strain with the greatest biomass fermentation capacity in step ii) is Kluyveromyces marxianus.

19. The process according to claim 17, wherein the strain with the greatest biomass fermentation capacity in step ii) is Kluyveromyces maxianus KJ830981.1.

20. A high protein biomass product produced from starch-containing cereal materials by a process using yeast strains, the process comprising the following steps:
i) producing glucose for use as a substrate from the starch-containing cereal materials by liquefaction and saccharification, including:
cleaning the starch-containing cereal materials and grounding;
carrying out liquefaction by raising the temperature to 85° C. and adjusting the pH to 5.5, then adding the enzyme α-amylase to carry out the liquefaction within two hours;
performing saccharification by lowering the temperature to 65° C. after two hours of liquefaction, and adjusting the pH to 4.5, then quantifying the α-glucoseamylase enzyme immediately after cooling and adjusting the pH to 4.5, before the saccharification process takes place;
conducting glucose content analysis by qualitative test using reagents such as iodine to determine the end of the saccharification process; and
separating glucose from the residue and stores the resulting sugars;
ii) selecting yeast strains which produce high protein biomass and storing the selected yeast strains, including:
selecting the yeast strains, which are capable of fermenting biomass, from the group consisting of S. cerevisiae, Pichia pastoris, C. utilis, Torulopsis, Geotrichum candidum, Kluyveromyces marxianus, Endomyces vernali, C. arbores, C. tropicalis, Torulopis utilis, Torulopis utilis var major, S. cerevisiae, Kluyveromyces marxianus, C. tropicalis, Monilia candia, Pichia pastoris, L. curvatus, L. reuteri, L. plantarum, L. parapentarum, and L. Pentosus;
culturing on a YPG agar medium including 10 g/L of yeast extract, 20 g/L of peptone and 20 g/L of glucose at 30° C., pH of 5.5, within the range of 18 to 24 hours, to refine each strain;
fermenting on the YPG agar medium including 10 g/L of yeast extract, 20 g/L of peptone and 20 g/L of glucose at 30° C., pH of 5.5, within 24 hours;
sampling for glucose analysis by High Performance Liquid Chromatography (HPLC);
selecting strains with the greatest biomass fermentation capacity when the residual glucose content is determined to be between 4-6 g/L;
genetic sequencing to identify the yeast strains based on gene maps; and
storing the selected strains;
iii) producing high protein biomass from the yeast strains of step ii) by using glucose produced in step i), including:
selecting yeast strains of step ii) which are propagated through three levels, level 1, level 2 and level 3, in the fermentation vessel in the corresponding medium, wherein:
level 1 and level 2 fermentation are carried out in the YPG medium including 10 g/L of yeast extract, 20 g/L of peptone and 20 g/L of glucose, at 30° C. and pH of 5.5;
level 3 fermentation is carried out in a medium including the glucose produced in step i) to produce a variety of cells having a minimum cell density of 10⁸ cells/mL for the propagation step in the fermentation stage;
wherein glucose is maintained at 200 g/L to optimize the fermentation; and
fermenting in which this step also goes through five levels of fermentation: level 1, level 2, level 3, level 4 and level 5, with the fermentation substrate source including the glucose produced in step i) at a concentration of 200 g/L; and
analyzing of glucose, sucrose and ethanol content in a fermentation solution after the fermentation process to determine the end of the process in order to obtain the most effective biomass, thereby producing the high protein biomass product.
